(19) United States
(12) Patent Application Publication
(10) Pub. No.: US 2009/0204142 A1
(43) Pub. Date: Aug. 13, 2009

(54) NASOLACRIMAL SYSTEM SURGICAL TOOL AND METHOD

(76) Inventor: Bruce B. Becker, Encino, CA (US)

Correspondence Address: CARSTENS & CAHOON, LLP, P O BOX 802334, DALLAS, TX, 75380, US

(21) Appl. No.: 12/302,269

(22) PCT Filed: May 25, 2007
(86) PCT No.: PCT/US2007/012461

§ 371 Date: Mar. 25, 2009

(30) Foreign Application Priority Data
May 26, 2006 (US) 11441558
Jun. 23, 2006 (US) 11473445

(51) Int. Cl. A61M 29/00

(52) U.S. Cl. 606/192

(57) ABSTRACT

A device and method for performing probing, irrigation, suction and intubation of the nasolacrimal system to treat for stenosis or obstruction includes a tubular probe (47) through which a tracer fluid is injected and collected in the nasal cavity to verify that the tip of the probe has passed through an obstruction and reached the nasal cavity. A sleeve (43) fitted over the probe has distal segment (44) that is inflated in order to retain the sleeve in the nasolacrimal system once the tubular probe has been withdrawn. Removal of blood and other obstructions encountered during the probing or intubation process is accomplished by connecting the proximal end of the probe to a suction device. An alternate embodiment provides a tool combining two elongated conduits, one of which is terminated at a distal end by an inflatable balloon. The other conduit is used for either “irrigating or suctioning. The tool is particularly useful to perform dacryocystoplasty (DCP) and dacryocystorhinostomy (DCR).

### FIELD OF THE INVENTION

The present invention relates to devices used for normalizing the flow of fluid in tubular organs of human bodies that have been injured by a disease or an accident. More specifically, the invention relates to probes, catheters, stents and drainage tubes used in treating canalicular and nasolacrimal duct stenosis, obstruction, lacerations or other trauma.

### BACKGROUND

The orbital portion of the lacrimal gland is located in the superotemporal orbit and the palpebral portion of the lacrimal gland is located on the posterior surface of the superotemporal upper lid. The lacrimal gland produces the aqueous portion of the tear film. Ductules from the orbital portion of the lacrimal gland pass through the adjacent palpebral lacrimal gland to empty in the superior conjunctival cul-de-sac. Smaller accessory lacrimal glands in the upper and lower lids also contribute to tear production. The tears bathe the surface of the eye and then drain into the puncta and canaliculi in the medial upper and lower lids. The superior and inferior canaliculi join as the short common canaliculus. The tears flow from the superior and inferior canaliculi through the common canaliculus, into the lacrimal sac, and down the nasal lacrimal duct into the nose.

The canaliculi can become obstructed or stenotic or otherwise damaged on a congenital basis or acquired as a result of some trauma such as lacerations, inflammation, side effects of chemotherapy, such as taxotere or five-fluorouracil—which may also affect the nasolacrimal duct—or the obstruction can be idiopathic. When the upper and lower canaliculi or the common canaliculus become obstructed, tears can no longer drain from the surface of the eye through the lacrimal system into the nose. The tears well up in the eye as a result, and run down the face. The excess tears blur the vision and the patient has to constantly dab the eye.

The nasolacrimal duct can be obstructed on a congenital basis occurring in about 2% to 6% of newborns. The acquired form of nasolacrimal duct obstruction can result from trauma, sarcoidosis or other diseases, but most commonly is idiopathic. When the nasolacrimal duct is obstructed, tears stagnate in the lacrimal sac and bacteria multiply therein leading to an infection of the lacrimal sac in many patients. The result is a painful enlargement of the lacrimal sac swollen with pus, and a discharge over the eye.

Canalicular obstruction or stenosis is usually treated by forming a new passage through the obstruction with a probe, also by dilatation with probes or with a balloon catheter. At times, a dacryocystorhinostomy (DCR) is performed. A DCR consists of the surgical creation of a new passageway from the lacrimal sac into the nasal cavity. This can be performed with a balloon catheter using an endoscope or externally through an incision. A silicon tube is often placed in the lacrimal system whether or not a DCR is performed.

In the case of trauma to the lacrimal drainage system, an intubation is performed to prevent scars from permanently clogging the canaliculi or nasolacrimal duct. In cases of canalicular or nasolacrimal duct obstruction from chemotherapy, intubation is performed as quickly as possible to prevent complete, irreversible closure.

A congenital nasolacrimal duct obstruction often resolves spontaneously, or with the use of antibiotic drops and massage of the lacrimal sac. However, a significant number of patients require surgical treatment. A probing is usually performed in these children. This is usually successful, if not balloon catheter dilatation is performed. Balloon catheter dilatation of the nasolacrimal duct or balloon catheter dacryocystoplasty (DCP) is also used as a primary procedure in children over twelve months of age, because the success rate of probing declines over a year of age. The procedure is also practiced in some case of acquired nasolacrimal duct obstruction.

The probes of the prior art are solid metal rods made of steel, bronze, silver or other metal. A flattened area in the center of the probe facilitates its manipulation.

Probing is performed by inserting a probe horizontally through the punctum and canaliculus into the lacrimal sac. The probe is then oriented vertically and pushed down the nasolacrimal duct into the nasal cavity. The surgeon must then confirm that the probe has penetrated all obstructions in the nasolacrimal duct and reached the nasal cavity. This is commonly done by placing a metal instrument into the nose and touching the probe. The surgeon feels for metal on metal contact indicating that the probe has entered the nasal cavity.

The probe is then removed from the lacrimal system. A syringe filled with fluorescein stained water with an attached short cannula is placed in the canaliculus and the fluid is irrigated through the lacrimal system into the nose. The fluid is recovered in the nose with a suction catheter. This confirms that the lacrimal system is patent after the probing. If the fluid does not irrigate into the nose, then the probing is repeated.

Probing in the lacrimal system presents several problems. The probe enters the nasal cavity through the opening of the nasolacrimal duct in the lateral nasal wall beneath the inferior turbinate. This area is difficult to access, making it often impossible for the surgeon to touch the probe in the nose with another instrument. In this event, the surgeon cannot confirm if the probe has entered the nasal cavity. Another problem is that irrigation of the lacrimal system is required to determine if the nasolacrimal duct obstructions have been opened. If irrigation through the lacrimal system down to the nasal cavity is not verified, the probing must be repeated. As a result, multiple procedures are performed that can cause repeated trauma to the lacrimal drainage system with each placement of a probe or cannula. In some resistant cases intubation is often needed. If silicone intubation needs to be performed, then the location and course of the nasolacrimal duct may need to be confirmed by probing before performing intubation of the lacrimal system.

The treatment of nasolacrimal duct obstruction in adults usually involves the creation of a new passage from the lacrimal sac directly into the nasal cavity according to a procedure called balloon catheter dacrocystorhinostomy (DCR) without attempting to dilate the nasolacrimal duct. That procedure involves a greater amount of bleeding that the balloon catheter DCP. In addition, the anterior middle turbinate may obstruct the outlet formed by the balloon DCR. Resection of the middle turbinate may be necessary leading to additional bleeding. The surgeon may extract the blood with a suction device through the nasal cavity and use an endoscope and attempt visualization with an endoscope. However this requires the surgeon to handle another instrument in addition to the endoscope. The suction may not be adequate to allow good visualization of the surgical site.

Bleeding in the lacrimal system or nose often occurs during the probing, intubation or associated procedures. The applicant is not aware of any expedient and practical device for clearing blood from the lacrimal system. Furthermore, the only known method for removing blood from the nasal cavity is by introducing into the nose a suction catheter through the naris. It is often difficult if not impossible to position the catheter in the inferior meatus in order to remove blood around the nose end entry into the nasolacrimal duct.

Intubation of the lacrimal system preferably with a silicon tube, is often performed after lacrimal surgery or as a primary treatment for nasolacrimal duct obstruction, canalicular stenosis, or canalicular laceration. The easiest device to insert is the Mini-monoka tube that consists of a silicon tube attached to a punctal plug. The tube is inserted through one canaliculus into the lacrimal sac. The plug on the proximal end of the tube is positioned at the punctum. The tube will thus stay in place without having to enter the nasolacrimal duct or the nose. Indeed, the Mini-monoka tube cannot generally be placed in the nasolacrimal duct or nose. If, however, intubation of the nasolacrimal duct is needed, then one of the two ends of the silicon tube is threaded through the canaliculus and down the nasolacrimal duct into the nose. The distal end of the tube, or of any probe attached to it, must be grasped in the nose and pulled into position. It can be very difficult to locate and grasp the tube in the nose of some patients. In some cases, it is impossible to find the tube. That is because the nasolacrimal duct empties into the nasal cavity in the inferior meatus beneath the inferior turbinate. U.S. Pat. No. 6,383,192 discloses a way to push an intubation device by means of a rod. However this method still require pulling the device out of the lacrimal duct from inside the nasal cavity. The nasolacrimal duct is very hard or impossible to visualize even with the help of a flexible endoscope. It is also very difficult to locate the duct simply by tactile sensation with an instrument. U.S. Pat. No. 6,878,165 Makino teaches another verification method involving the insertion of a miniature light at the tip of a probe or stent. The illumination of the nasal cavity offers visual proof that penetration is complete, unless, as is usually the case, the light is blocked by an edema or an accumulation of blood.

When treating nasolacrimal duct obstruction with balloon catheter dilatation, a deflated balloon catheter is introduced through a punctum into one of the canaliculi, the common canaliculus, the lacrimal sac, and down the nasolacrimal duct into the nose. The surgeon must then confirm that the catheter has penetrated all obstructions and entered the nasal cavity. He must also insure that the catheter has not opened a false passage by piercing through the wall of the nasolacrimal duct instead of going down the duct into the nose.

Confirmation of the presence of the catheter into the nasal cavity is rendered difficult by the anatomy of the area. The nasolacrimal duct empties into the nose on the lateral nasal wall beneath the inferior turbinate. It is often very difficult if not impossible to observe the nasolacrimal duct and the entry of the catheter into the nose cavity. Edema of the inferior turbinate and nasal mucosal bleeding can obstruct visualization. The surgeon usually introduces a metal instrument through the external naris into the nose and tries blindly to touch the tip of the balloon catheter until a contact between the instrument and the catheter is felt. Alternatively, the surgeon may attempt to visualize the catheter with an endoscope or a headlight; a procedure with which many ophthalmic surgeons are not very familiar.

The balloon catheter is then connected to an inflation device, and its expansion dilates the duct. After deflation the catheter is pulled out of the nasolacrimal network. A syringe equipped with a short cannula is used to irrigate a tracer fluid through the lacrimal system into the nose. Recovery of some of the fluid in the nose by means of a suction device confirms that the lacrimal system is patent. If no tracer fluid is recovered, the balloon catheter has to be reintroduced in a slightly different orientation or pushed further down the nasolacrimal duct, and inflation repeated. After which a new-irrigation confirming procedure must be performed. These repeated procedures often cause multiple traumas to the lacrimal drainage system.

The instant invention results from attempts to avoid the aforesaid problems and provide more efficient, simpler and safer procedures in the treatment of nasolacrimal duct obstructions, including improved probe use and function, and providing improved irrigation, dilatation, and/or intubation.

### SUMMARY

The instant embodiments provide devices and method to better treat obstructions in the nasolacrimal system.

Some embodiments provide a simple and practical method for verifying that a nasolacrimal system probe or intubation sleeve has been inserted through all obstructions down to the nasal cavity. In some embodiments the new probe comprises a tube, shaped and dimensioned to penetrate a patient's canaliculus and nasolacrimal duct. In some embodiments the tube has an axial lumen through which a tracing fluid can be injected. In some embodiments recovery of the fluid in the nasal cavity indicates that the probe has passed through any obstructions in any part of the system.

Some embodiments offer a novel method of intubation of the nasolacrimal system using a sleeve that fits over the aforesaid probe and can be threaded all the way down the nasal cavity through a patient's punctum, canaliculus, lacrimal sac and nasolacrimal duct. The tip of the sleeve can be inflated to stabilize its position before the probe is withdrawn. The probe can be used to irrigate the nasolacrimal system with a tracing fluid which once recovered through a suction apparatus in the nasal cavity provides a positive indication that the sleeve is in place.

A version of the probe can be adapted to suction blood, and other fluids from the nasolacrimal duct and tracing fluids from the nasal cavity.

In some embodiments there is provided a device for the treatment of a patient's canaliculus and nasolacrimal duct stenosis which comprises: a tube shaped and dimensioned to penetrate said canaliculus and duct; said tube being made of a substantially hard material, and having a proximal end, a blunted distal end, an axial lumen, a total length between approximately 4 and 50 centimeters and an outer diameter between 0.125 and 4.00 millimeters; a connector at said proximal end; said lumen having at least one orifice at said distal end.

In some embodiments said orifice comprises at least one radial outlet about 0.5 to 30 millimeters from said distal end, and said outlet has a diameter between about 0.025 and 2.5 millimeters. In some embodiments said material substantially is taken from a group consisting essentially of stainless steel, bronze, silver, aluminum, titanium, brass, and alloy thereof, Kevlar, Nitinol, polymide, Dacron, nylon, EPTFE and PVC; and said tube further comprises a slanted radial flange proximate to said connector. In some embodiments the device further comprises a flexible catheter having a distal end shaped and dimensioned to interlock with said connector, and a proximal end shaped and dimensioned to interlock with a syringe. In some embodiments said connector is shaped and dimensioned to interlock with a syringe. In some embodiments the device further comprises a stiffening rod diametrically sized to engage said lumen, and having a length at least equal said total length. In some embodiments said rod has an enlarged manipulable end section. In some embodiments the device further comprises a flexible sleeve having a proximal end, a distal end, an axial interior channel closed at said distal end and being dimensioned to allow said channel to be engaged by said tube, and a length shorter than said total length of said tube. In some embodiments said flexible sleeve has a radial hole proximate said distal end. In some embodiments said tube comprises a radial flange proximate to said connector, and wherein said sleeve comprises a first radial flange around said proximal end; said first radial flange being oriented at the same axial angle as the radial flange of said tube. In some embodiments said sleeve comprises a second radial flange distally proximate to said first radial flange. In some embodiments said sleeve further comprises a first sealing implement across said channel, proximate said proximal end. In some embodiments said sleeve further comprises an inflatable segment between said radial hole and said distal end. In some embodiments said sleeve further comprises a second sealing implement across said channel at a short proximal distance from said segment. In some embodiments said inflatable segment comprises said sleeve having a reduced wall thickness along said segment.

Some embodiments provide a method for probing the integrity of a patient's canaliculus and nasolacrimal duct which comprises the steps of: inserting the device of some embodiments through the patient's punctum and canaliculus down the lacrimal sac; tilting the device about 90 degrees into alignment with the nasolacrimal duct; pushing the device through the nasolacrimal duct down to the nasal cavity; injecting a tracing fluid through said connector; and recovering part of said fluid from the nasal cavity; whereby recovery of a trace of said fluid confirms that the device has penetrated all obstructions and entered the nasal cavity.

Some embodiments provide a method for intubating a patient's nasolacrimal duct which comprises the steps of: inserting the metallic tube and the sleeve of some embodiments through a patient's punctum, canaliculus into the lacrimal sac; tilting the sleeve and tube about 90 degrees into alignment with the patient's nasolacrimal duct; pushing the tube and sleeve through the nasolacrimal duct down to the nasal cavity; injecting a tracing fluid into the tube; verifying that the tube and sleeve have reached the nasal cavity by recovering traces of said fluid in said cavity; and withdrawing said tube from said sleeve.

Some embodiments provide a method for intubating a patient's nasolacrimal duct which comprises the steps of: inserting the tube and the sleeve of some embodiments through a patient's punctum, canaliculus into the lacrimal sac; tilting the sleeve and tube about 90 degrees into alignment with the nasolacrimal duct; pushing the tube and sleeve through the nasolacrimal duct down to the nasal cavity; injecting a volume of fluid through said connector sufficient to inflate said inflatable segment; partially withdrawing said tube from said sleeve by a distance sufficient to bring said outlet between said proximal end of the sleeve and said sealing implement at a short proximal distance from the inflatable segment; injecting a tracing fluid into the tube; verifying that the tube and sleeve have reached the nasal cavity by recovering traces of said fluid in said cavity; and withdrawing said tube from said sleeve.

Some embodiments provide a method which further comprises inserting a stiffening rod diametrically sized to engage said lumen and having a length greater than said total length into said tube, prior to insertion of said tube into said sleeve. Some embodiments provide a method which further comprises inserting a stiffening rod diametrically sized to engage said lumen and having a length substantially greater than said total length into said tube prior to insertion of said tube into said sleeve. Some embodiments provide a method which further comprises connecting said tube to a suction device during said step of pushing. Some embodiments provide a method which further comprises connecting said tube to a suction device during said step of pushing. In some embodiments said step of recovering comprises connecting a suction device to said tube. In some embodiments said step of recovering comprises connecting a suction device to the proximal end of said tube. In some embodiments said step of pushing further comprises pushing said second flange inside said punctum and resting said first flange against the external rim of said punctum.

The instant invention results from attempts to achieve intubation without having to retrieve the end of the tube inside the nose, to perform probing and irrigation in a single step, to expediously clear blood and other fluids from the nasal cavity and the nasolacrimal duct.

In some embodiments there is provided a multi-conduit dilating balloon catheter designed to be inserted into a patient nasolacrimal system which incorporates an irrigation catheter that allows immediate injection of a tracer fluid without having to retract the catheter. In some embodiments, retrieval of the fluid in the nasal cavity provides confirmation that all obstructions have been penetrated without false passage of the catheter through the wall of the lacrimal network. The combination catheter can also be run through a new passageway pierced with a probe through the inferomedial wall of the lacrimal sac and the lateral nasal wall.

In some embodiments, the multi-functional surgical tool for the treatment of nasolacrimal duct obstruction comprises a first elongated tubular conduit having proximal and distal ends, a second elongated tubular conduit having proximal and distal extremities. The second conduit can be parallelly coupled to the first conduit. In some embodiments, an inflatable balloon is provided at a distal end of the first conduit. The surgical tool can be sized to be introduced into a patient's nasolacrimal duct through one of the patient's canaliculi.

In some embodiments, the coupled conduits have a total maximum cross-sectional dimension ranging between about 1.0 millimeter and 2.5 millimeters, and can be coaxial, wherein the second conduit comprises a median portion running inside the first conduit and balloon, a proximal section exiting through an opening in the first conduit, and a distal section exiting through an aperture beyond the balloon.

In some embodiments, the opening in the first conduit is sealed around the proximal section and the aperture is sealed around the distal section of the second conduit.

In some embodiments, the tool further comprises a first connector at the proximal end of the first conduit and a second connector at the proximal extremity of the second conduit.

In some embodiments, the tool also comprises an irrigation device connected to the second connector. Alternately, a suction device may be connected to the second connector.

In some embodiments, the tool further comprises an inflating device connected to the first connector.

In some embodiments, there is provided a modified version of the tool where the second conduit has an angled distal part.

In some embodiments, there is provided a surgical tool which combines a balloon catheter and an irrigation conduit coupled together in an elongated instrument which is sized to be manually introduced into a patient's nasolacrimal network, wherein an inflatable balloon and a discharge outlet of the irrigation conduit are located at the distal end of the instrument.

In some embodiments, there is provided a method for treating an obstruction in a patient nasolacrimal system, which comprises: inserting into said system a tool combining a balloon catheter and an irrigation catheter, joined parallelly to each other; inflating said balloon catheter; and injecting a tracer fluid through said irrigation catheter.

In some embodiments, the method further comprises detecting an amount of said tracer fluid beyond said obstruction.

In some embodiments, the method further comprises successively inflating and deflating said balloon catheter, and performing a plurality of said injecting steps.

In some embodiments, the method further comprises suctioning blood out of the nasolacrimal system through the irrigation catheter, and injecting medication in the system through said irrigation catheter.

In some embodiments, the method further comprises piercing a passageway through the patient's inferomedial wall with a probe and running the tool through said passageway.

### BRIEF DESCRIPTION OF THE DRAWINGS

FIG. 1 is a cross-sectional view of a nasolacrimal probe according to an embodiment of the invention.

FIG. 2 is a cross-sectional view of an intubation sleeve.

FIG. 3 is a partial cross-sectional view of a combination of the aforesaid probe and sleeve.

FIG. 4 is a partial cross-sectional view of a combination of the aforesaid probe and sleeve having a reinforcing distal internal bushing inserted in the sleeve.

FIG. 5 is a partial cross-sectional view of an alternate embodiment of the sleeve in the inflated position.

FIG. 6 is a cross-sectional view of the alternate embodiment of the sleeve in the irrigating position.

FIG. 7 is a perspective view of the proximal end of the probe and sleeve combination.

FIG. 8 illustrates the first positioning of the probe.

FIG. 9 illustrates the final position of the probe.

FIG. 10 illustrates intubation with a sleeve having an inflatable end segment.

FIG. 11 illustrates suction through the probe.

FIG. 12 is a cross-sectional view of an exemplary embodiment of a multi-conduit surgical tool according to the invention.

FIG. 13 is a diagrammatical illustration of the use of the tool in a DCP.

FIG. 14 is a diagrammatical illustration of the use of the tool in a DCR.

FIG. 15 is a diagrammatical illustration of the use of the tool in a transnasal DCR.

### DESCRIPTION OF THE EXEMPLARY EMBODIMENTS

Referring now to the drawing, there is shown in FIG. 1 a cross-sectional view of nasolacrimal irrigating probe 1 specifically designed to probe obstructions in a patient's drainage system and nasolacrimal duct. The device comprises a tube 2 having a blunted distal end 3, an open proximal end 4 equipped with a luer-lock 5 or other type of connector, and axial lumen 6. An outwardly projecting radial flange 7 near the luer-lock is slanted at an angle from about 20 to 90 degrees, and can typically be 45 degrees to the axis of the probe. The probe can also be made without the flange. The device 1 is shaped and dimensioned for insertion through a patient's punctum and canaliculus, then through the lacrimal sac and into the nasolacrimal duct down to the nasal cavity down to the point where the flange 7 rests against the entry punctum. The device has a slight degree of flexibility resulting from the choice of material and its dimensions. The tube 2 and connector 5 are preferably made of a metal such as stainless steel, titanium, silver, aluminum, bronze, brass or any alloy of these metals, or of synthetic materials such as Kevlar, Nitinol, polymide, Dacron, nylon, EPTFE or PVC. The total insertable length A is preferably 10 centimeters, but may fall between approximately 5 and 50 centimeters. The outer diameter B of the tube, is preferably 0.64 millimeters, but may range from approximately 0.10 to 3.75 millimeters.

One or a pair of diametrically opposite radial orifices or outlets 8 are located 0.5 to 30 millimeters approximately from the distal end 3. The diameter of each outlet is preferably 0.025 millimeters, but can reach 2.5 millimeters in large models. The distal end is blunted by a rounded or conical tip 9. Alternately, a single axial orifice with a rounded lip to prevent abrasion may be provided at the distal end of the tube with a diameter of, preferably 0.25 millimeters, but could fall anywhere between 0.025 and 2.5 millimeters. Although the tube is made of a rigid or semi-rigid material, its length and the relative thinness of its wall may render it quite flexible and easily bendable. To avoid damaging the tube and generally increase its rigidity, a stiffening rod 10 diametrically sized to engage the lumen 6 of the tube is provided. The rod has a length slightly in excess of the total length A of the tube, and is made of the same type of material. A manipulable flattened or otherwise enlarged section 10a at the proximal end of the rod facilitates its handling.

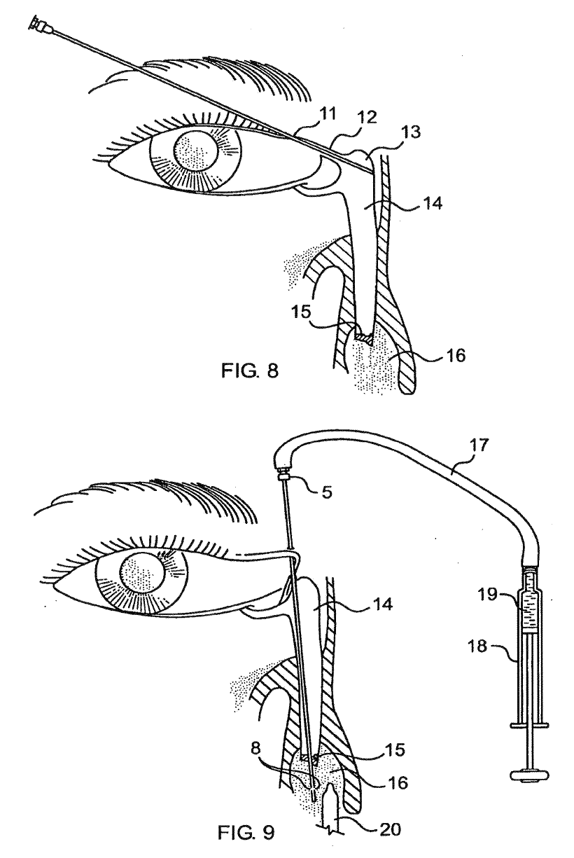

Probing of the nasolacrimal duct with the device 1 begins with inserting the tube through a patient's punctum 11 and canaliculus 12 down to the lacrimal sac 13, as shown in FIG. 8. A barrier is felt when the probe encounters the medial lacrimal sac wall and lacrimal fossa. At that point, the probe is then retracted about 0.5 millimeters and is tilted about 90 degrees into alignment with the nasolacrimal duct 14 as illustrated in FIG. 9. The probe is pushed down the nasolacrimal duct through any obstruction 15 and into the nasal cavity 16. A flexible conduit 17 is connected at one end to the connector 5 and at the other end to a syringe 18 loaded with fluorescein or methylene blue stained fluid 19 or any other colored liquid or gas tracer. Alternately the syringe may be applied directly to the connector 5. The fluid is injected to irrigate through the probe into the nose. The fluid flows out of the outlets 8 into the nasal cavity. Traces of the fluid can be recovered in the nose with a suction catheter 20. A lack of fluid recovery in the nose indicate that the probe has not penetrated all obstructions and reached the nose. The surgeon can then push with greater force or pull the probe back slightly and drive it into the nasal cavity at a slightly different angle. Detection of the tracer fluid into the nose is a positive indication that all obstructions have been cleared, and no divergent passage through tissues surrounding the nasolacrimal duct has been opened by the probe.

It should be noted that the surgeon does not have to perform the difficult and sometime impossible task of touching the tip of the probe in the nose with another metal instrument to confirm that the probe has duly entered the nasal cavity. Furthermore, the irrigation does not have to be performed as a second procedure after a solid probe of the prior art has been withdrawn from the lacrimal system. No second probing needs to be done if the irrigation is not successful.

The stiffening rod 10 must be withdrawn before the flexible tube or syringe is connected to the probe. Preferably, the rod is used when the probe encounters an obstacle and cannot readily and safely be pushed through it.

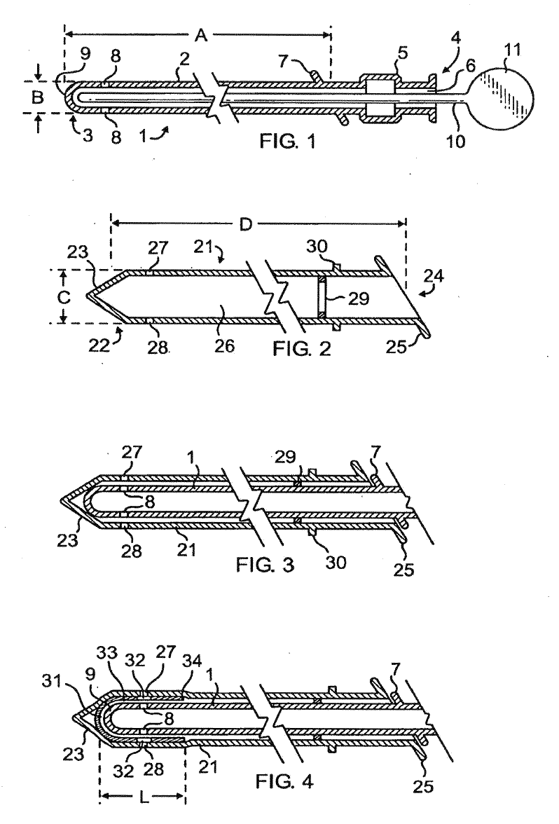

Referring now to FIG. 2, there is shown an intubation sleeve 21 shaped and dimensioned to be used in connection with the above-described probe 1. The outer diameter of the sleeve is preferably 1.125 millimeters, but could fall between 0.25 and 4.0 millimeters. The sleeve is flexible and preferably made of silicone, polypropylene or other medically approved synthetic material. The distal end of the sleeve 22 is preferably closed by a conical tip 23 or a rounded one substantially similar to the tip 9 of the probe. The proximal end 24 of the sleeve is open and is surrounded by a first outwardly projecting radial flange 25 that is oriented at an angle between 20 to 90 degrees and preferably approximately 45 degrees with the axis of the sleeve like the flange 7 of the probe. The internal channel 26 of the sleeve is dimensioned to be loosely engaged by the probe 1 as shown in FIG. 3 so that a fluid injected into the probe can readily exit the radial holes 8 or the axial orifice at the distal end of the probe and flow freely into the sleeve. The insertable length D of the sleeve is about 3 millimeters shorter than the insertable length A of the probe.

A hole 27, or alternatively two diametrically opposite radial holes 27, 28, bored through the sleeve at approximately the same distance from the tip 23 as the distance between the outlets 8 of the probe are from its tip 3, let tracing or irrigating fluid injected into the probe escape from the sleeve into the nasal cavity. An O-ring, self-sealing diaphragm 29, or other type of sealing implement located between 0 and 100 millimeters and preferably about 3 millimeters from the proximal end 24 of the sleeve can be penetrated by the probe and maintain a hermetic barrier that will prevent any fluid in the channel 26 from leaking through the proximal end 24 of the sleeve. A second radial flange 30 distally proximate to the first flange 25 is designed to lie just inside the punctum to keep the first flange resting against the edge of the punctum. The second flange can have an oval shape, and have a maximum exterior diameter between 0.3 and 6 millimeters, preferably 2.5 millimeters, and is preferably orthogonal to the axis of the sleeve.

Intubation of the nasolacrimal duct is performed by first inserting the probe 1, and optionally its stiffening rod 10, into the sleeve 21 until the tip of the probe touches the closed distal end of the sleeve as shown in FIG. 3. The combined probe and sleeve are then threaded through a patient's punctum, canaliculus, lacrimal sac, nasolacrimal duct all the way down to the nose in the same manner as described above and illustrated in FIGS. 8 and 9 in connection with the probe, until the second flange 30 is set into the patient's punctum and the first flange 25 rests against the external rim of the punctum.

The surgeon may encounter resistance when pushing the second radial flange 30 of the sleeve through the punctum into the proximal canaliculus if the punctum is somewhat small in diameter. The distal end of the probe will exert pressure upon the very distal end of the sleeve if the surgeon applies a large amount of force on the probe while attempting to push the second radial flange 30 through the punctum. However, puncture of the distal end of the sleeve is prevented by the slanted flange 7 of the probe coming into contact with the slanted flange 25 of the sleeve. This stops further penetration of the probe into the sleeve, while allowing the surgeon to apply pressure on the probe and sleeve assembly in order to push the second flange 30 of the sleeve through the punctum.

Alternately, as shown in FIG. 4, in order to further prevent puncture of the distal end of the sleeve 21 by the probe during the intubation procedure, an internal reinforcing sheath 31 can be inserted into the sleeve to be located between the tip 9 of the probe 1 and the distal end tip 23 of the sleeve. The sheath has a generally closed-ended, hollow tubular shape having an open proximal end 34, a closed, opposite distal end, and substantially cylindrical sidewalls defining inner well 33 sized to accept the tip of the probe therein. The sheath is preferably made from a material which is more puncture resistant than the material of the sleeve. Examples of such materials include polypropylene and polyether block amide (PEBA) which is commercially available under the brand name PEBAX from the Arkema company of Paris, France, or other formable biocompatible, puncture resistant materials known to those skilled in the art. Because such materials tend to be less flexible, the sheath preferably has a length L which is significantly shorter than the insertable length D of the sleeve.

In order for the sheath 31 to be retained within the inner distal end of the sleeve 21, the outer diameter of the sheath can be larger than the unstressed inner diameter of the sleeve thereby causing a friction fit between them, keeping the sheath lodged and in a stabilized position relative to the sleeve. Therefore, in this embodiment the outer diameter of the sheath is preferably 0.85 millimeters (0.033 inch), but could fall between 0.25 (0.01 inch) and 13 millimeters (0.5 inch). The length of the sheath is preferably about 15 millimeters, but could fall between about 1 millimeter and 250 millimeters. The internal well 33 of the sheath is dimensioned to be loosely engaged by the probe 1 as shown in FIG. 4 so that a fluid injected into the probe can readily exit the radial holes 8 or the axial orifice at the distal end of the probe and flow freely into the sleeve. The inner diameter of the sheath is preferably 0.635 millimeter (0.025 inch), but could fall between 0.25 (0.005 inch) and 12.6 millimeters (0.497 inch).

During manufacture, the sheath 31 can be pushed into place inside the sleeve 21 by an elongated implement dimensioned similarly to the probe. A lubricant can be used to coat the outer surface of the sheath to facilitate emplacement. The sheath has radial holes 32 in its sidewall corresponding to and in alignment with the outlets 8 in the probe and holes 27,28 in the sleeve. Care should be taken to ensure that the holes are aligned during manufacture. One way to do so would be by forming the holes and outlets after the sheath has been mated to the sleeve such as through a drilling step.

Alternately, the sheath can be formed into a mesh structure and made from a malleable material such as stainless steel. During manufacture, the sheath can be inserted into the sleeve in a collapsed state and then expanded to bear against and be held in place by a friction fit with the internal walls of the sleeve. Such a mesh structure can obviate the need for aligning the holes of the sleeve and sheath. Alternately, the sheath can be formed by a flexible, puncture resistant fabric weave mounted upon a rigid supporting ring formed into the open end 34 of the sheath to keep it open.

The distal tip of the reinforcing sheath may be thicker than the walls of the sleeve so that even greater puncture resistance is provided. Therefore, in this embodiment the thickness of the sheath is preferably 0.20 millimeters (0.008 inch), but could fall between 0.20 millimeters (0.008 inch) and 13 millimeters (0.5 inch).

If the internal diameter of the sleeve closely matches the external diameter of the probe, irrigation may be facilitated by aligning the outlets with the holes, as shown in FIG. 7. A mark 31 along the external wall of the probe that is aligned with one of the outlets 7, 8 is brought to match an indicium 42 on the flange 25 of the sleeve 21 that is aligned with one of the holes 27, 28.

After the presence of the sleeve and probe in the nasal cavity has been verified by the collection of some of tracing liquid in the nasal cavity, the probe is withdrawn leaving the sleeve in place.

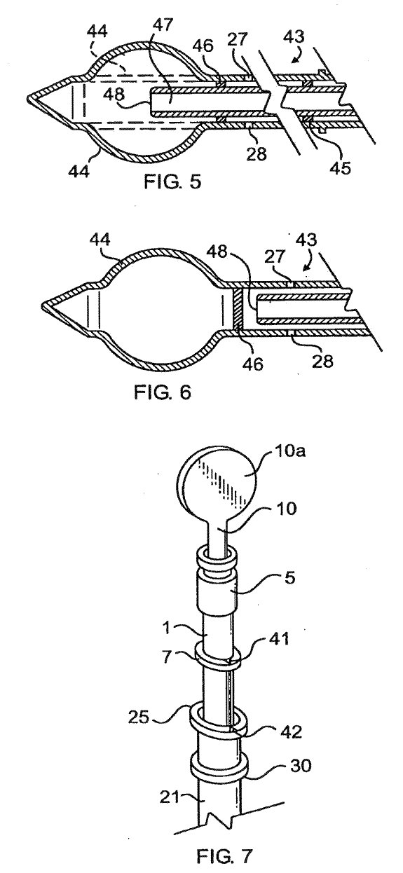

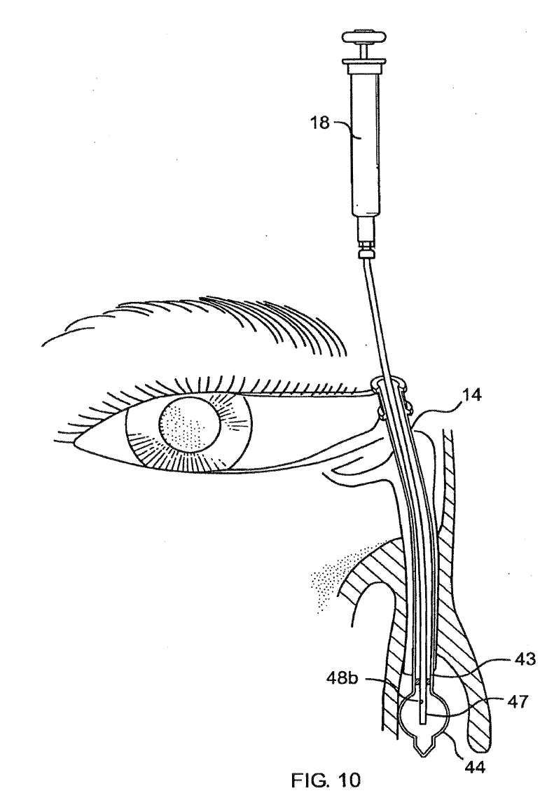

In an alternate version 43 of the sleeve illustrated in FIGS. 5 and 6, an inflatable segment 44 is formed near the distal end of the sleeve. The inflatable segment is preferable implemented using a resiliently expandable material, or by a reduction in the thickness of the sleeve wall slightly distally from the radial holes 27 and 28 in order to create a resiliently expandable balloon under internal pressure. Alternatively a segment made of easily expanded material can be attached to the distal end of a non-expandable sleeve. The entire sleeve can also be made of easily expanded sheet material. A first O-ring, self-sealing diaphragm 45 or other self-sealing implements may optionally be positioned between the proximal end of the sleeve and the radial holes, preferably at a short distance from the proximal end of the sleeve. A second self-sealing implement 46 is positioned between the radial holes 27, 28 and the inflatable segment 44. A fluid can be injected through a probe 47 having an axial orifice 48 or, alternately, at least one radial orifice 48b at its distant end, after the probe has been used to push the sleeve into position through the patient's punctum, canaliculus, lacrimal sac and nasolacrimal duct into the nasal cavity with the open tip of the probe resting in or just past the inflatable segment. The injection of the fluid causes the inflatable segment to bellow out and positively lock the sleeve in position as illustrated in FIG. 10. The probe is then withdrawn to a distance sufficient to place the orifice 48 between the first 45 and the second 46 self-sealing implements as shown in FIG. 6. Additional injection of tinted fluid will cause the fluid to escape into the nasal cavity through the axial orifice 48 into the sleeve. The second self sealing implement 46 prevents fluid from leaking out of the inflated area 44, thus maintaining the inflation. The first self sealing implement 45 prevents leakage of the fluid out of the proximal end of the sleeve, causing the fluid to exit through the radial holes 27, 28. Once the correct positioning of the sleeve has been verified through the collection of tracing fluid in the nasal cavity, the probe can be withdrawn while the sleeve distal segment remains inflated keeping the sleeve safely in place. In both cases, the sleeve can be later removed by grasping the flange 25 and pulling the sleeve out of the lacrimal system. Prior to removal, the end segment 44 can be deflated by pushing the probe through the second self sealing implement 46, and letting the fluid escape into the nasal cavity or suctioning it through the probe as explained below. Otherwise, the fluid will be allowed to slowly leak out of the sleeve on its own, whereupon the sleeve can be removed days, weeks or even months later.

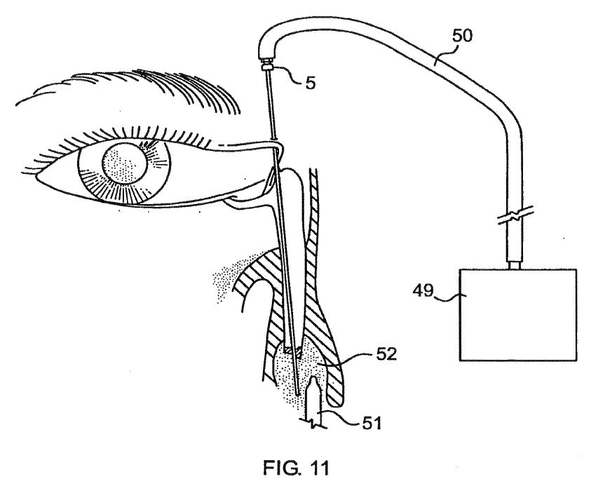

Each of the probes 1, 37 can be used for suctioning blood from the lacrimal system or nasal cavity caused by the probing or intubating process, as well as for suctioning the tracer fluid from the nasal cavity as illustrated in FIG. 11.

At the end of the probing or intubation procedure or after having been pushed through the lacrimal system as described above, the probe with or without either of the sleeves 21, 43 is connected to a suction device 49 by way of a catheter 50. Suction is then performed to either retrieve the tracer fluid out of the nasal cavity or to remove blood caused by abrasion during the procedure. The suction device may also be connected and activated during the insertion process of the probe or probe-and-sleeve combination through the nasolacrimal system in order to suction any obstructive tissue or blood. After installation of the probe or probe-and-sleeve combination, a tracer fluid may be injected with a syringe or eye dropper 51 through the nares 52. The fluid is then retrieved through the probe connected to the suction device to confirm proper placement of the sleeve or that the probe has reached the nasal cavity.

It can thus be seen that the tubular probe of the invention is a very versatile instrument that can be used not only for probing the nasolacrimal ducts, but also to perform intubation, irrigation and even suction of obstructive material.

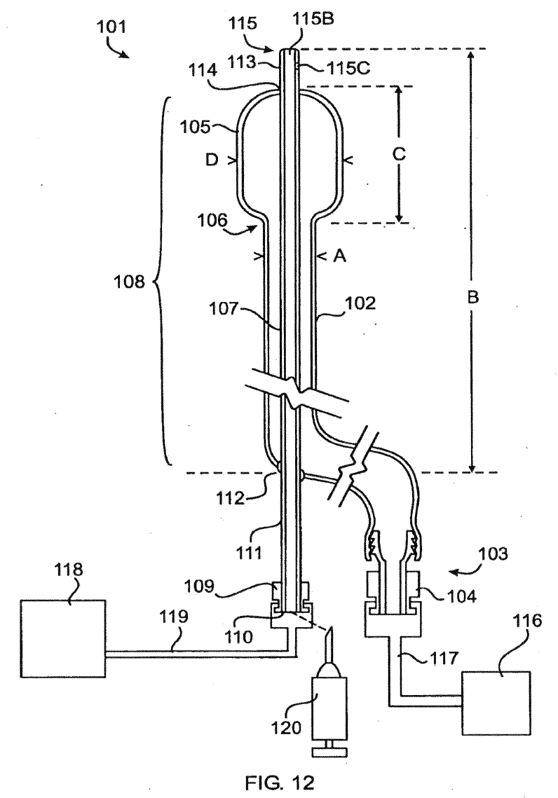

Referring now to FIG. 12, there is shown a multi-functional surgical tool 101 for the treatment of a nasolacrimal obstruction. The tool comprises a first elongated conduit 102 having at a proximal end 103 a luer-lock 104 or other type of connector, and an inflatable balloon 105 at a distal end 106. A second elongated conduit 107 is parallelly coupled to the first conduit. In this case, a median section 108 of the second conduit runs near coaxially within the first conduit 102. A second luer-lock or other type connector 109 is attached to the proximal extremity 110 of the second conduit. The proximal section 111 of the second conduit exits the first conduit through an opening 112 in the wall of the first conduit. A distal portion 113 of the second conduit exits through an aperture 114 beyond but near the inflatable balloon 105. While the distal end 6 of the first conduit is closed by the inflatable balloon 105, the second conduit has a blunt distal end 115 and an axial port 115B. In addition, one or more radial ports 115C through a sidewall of the distal portion 113 and thereby near the distal end 115 provide a fluid passageway should the axial port be blocked. The opening 112 and aperture 114 are hermetically sealed around the second conduit.

It would be an obvious modification to run the first conduit within the second one with the inflatable balloon being positioned beyond the distal end of the second conduit. The two conduits could also run contiguously rather than coaxially.

The tool is sized to be introduced into a patient's nasolacrimal duct through one of said patient's canaliculi. The total cross-sectional diameter A of the combined conduits has a maximum dimension of between about 0.1 and 10 millimeters, and typically about 1 millimeter for the present embodiment and about 2.5 millimeters for a transnasal embodiment described below. The total insertable length B of the tool may be within range from about 1 to 50 centimeters, and is typically about 15 centimeters. The inflatable balloon 105 has a length C between about 0.5 and 5 centimeters, and an inflated cross-sectional diameter D of up to about 2 centimeters. The balloon portion is preferably made of a resiliently expandable synthetic material such as polyethylene terephthalate (PET), latex, silicon or other elastomeric material. It can also be made of nylon, polyurethane, polyvinyl chloride, cross-linked polyethylene, polyolefins, HPTFE, HPE, HDPE, LDPE, EPTFE, and block polymers. The remainder of the first conduit is preferably made of polyethylene terephthalate (PET).

The second conduit 7 is preferably made of an hard but flexible material such as stainless steel and has enough rigidity to be pushed through obstructions in the nasolacrimal network, but is flexible enough to bend around small curves. Other metals and alloys such as titanium, silver, aluminum, bronze, brass, and synthetics like Kevlar and Nitinol may also be suitable.

During use, the first connector 104 at the proximal end of the first conduit is connected to an inflation device 116 by means of an appropriate connecting tube 117.

The second connector 109 at the proximal end of the second conduit is connected to a suction device 118 through an appropriate connecting tube 119 or, at other times, to a syringe 120 or other irrigation devices. In this way the port can act as an entry port for suctioned material or as an exit port for irrigation fluid.

The above-described surgical tool can be used in a variety of surgical interventions as explained below.

In a balloon catheter DCP procedure, the surgeon begins dilating the punctum with a punctal dilator before inserting a probe through the punctum and canaliculus down to the lacrimal sac. A barrier is felt when the probe encounters the medial lacrimal sac wall and lacrima fossa. The probe is then retracted about 0.5 millimeters and is tilted about 90 degrees into alignment with the nasolacrimal duct. The probe is pushed down the nasolacrimal duct and into the nasal cavity, then removed.

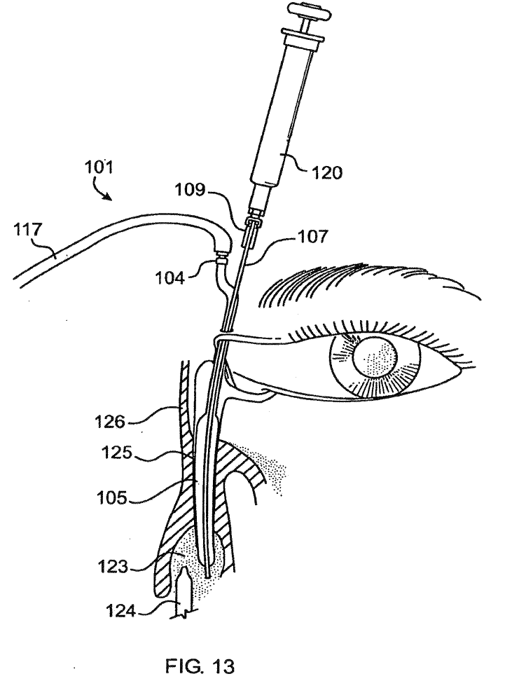

The tool 101, with the balloon 105 deflated, is inserted in the same manner as the probe down to the nasal cavity 123 as illustrated in FIG. 13. The syringe 120 can be connected directly to the luer-lock connector 109 of the second conduit 107. Alternatively, the flexible tube 119 is connected at one end to the luer-lock connector 109 and at the other end to the syringe 120 loaded with fluorescein stained fluid or any other tracing fluid. The fluid is injected to irrigate through the second conduit 107 into the nose. Traces of the fluid can be recovered in the nose with a suction device 124. A lack of fluid in the nose indicates that the tool has not penetrated all obstructions and reached the nose. The surgeon can then push with greater force or pull the tool slightly and drive it into the nasal cavity at a slightly different angle. Detection of the tracing fluid in the nose is a positive indication that all obstructions have been penetrated.

It should be noted that the surgeon does not have to perform the difficult and sometime impossible task of touching the tip of the tool in the nose with another metal instrument in order to confirm that the tool has duly entered the nasal cavity.

The tube 117 from the inflation device 116 is connected to the luer-lock connector 104 at the proximal end of the first conduit. An inflating fluid is sent down that conduit to inflate the balloon 105 and dilate the stenoic nasolacrimal duct 125. The balloon is deflated and pulled more proximally before a new inflation cycle is performed. The procedure is repeated as many times as it may be necessary to dilate the entire duct and the sac-duct junction. An irrigation cycle is again performed. If irrigation is not successful the tool may be pushed back before repeating the inflation procedure until tracer fluid recovery in the nose confirms that all obstructions have been corrected.

If significant bleeding occurs during the procedure, the syringe 120 is removed and the tube 119 is connected to the suction device 118 in order to remove the blood.

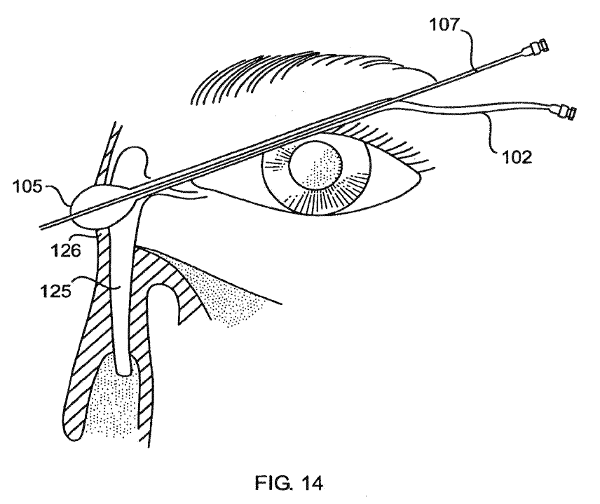

In a balloon catheter DCR, a probe is inserted as described above in connection with a DCP into the lacrimal sac. It is the pushed through the inferomedial wall of the sac, lacrimal fossa, and lateral nasal wall into the nose. The probe is visualized endoscopically. The probe is further pushed through multiple adjacent areas to enlarge the opening and push bone chips of lacrimal fossa bone and possibly ethmoid bone into the nasal cavity. The probe is withdrawn, and the tool 101 is pushed through the superior or inferior canaliculus, the common canaliculus, the lacrimal sac and through the prepared opening in the inferomedial lacrimal sac wall, lacrimal fossa, lateral nasal wall into the nasal cavity. A syringe is used to inject a tracer fluid through the second conduit 107 as shown in FIG. 14. The presence of tracer fluid in the nose is confirmed. The syringe is then replaced by the suction device 118, and blood and tissue debris are suctioned. Bleeding is usually more profuse than in a DCP and suctioning may have to be performed during the entire operation.

Inflation of the balloon 105 by the inflation device 116, through the tube 117 and the first conduit 102 is performed as described earlier including about the opening into the inferomedial wall 126 as shown in FIG. 14 and lateral nasal wall. The tool in then withdrawn.

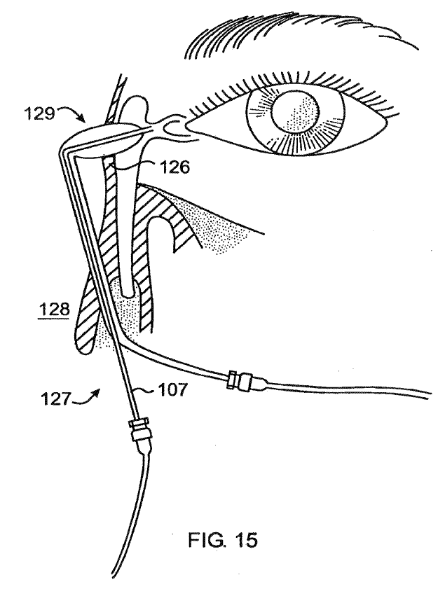

The performance of a transnasal balloon catheter DCR is performed basically in the same manner as a above, except that after probing and piercing of the inferomedial wall and lateral nasal wall, a modified tool 127 is brought up through the external naris 128 up the nasal cavity and pushed through the opening in the inferomedial wall 126 of the lacrimal sac into the lacrimal sac as shown in FIG. 15.

The modified tool has a distal segment 129 bent at an angle of between about 10 and 170 degrees, and is typically about 90 degrees.

A suction procedure is performed through the second conduit 107, then a dilatation procedure as described above. After disconnecting the suction device 118, irrigation may be performed.

The second conduit 107 can also be used at that time to deliver medications into the nasolacrimal duct.

After having removed the tool 127, the surgeon inserts a short cannula of about 1.0 centimeter in length into the punctum and canaliculus. With syringe, a tracer fluid is injected through the cannula into the nasolacrimal network. If none of the fluid is recovered in the nose, the procedure must be repeated.

In lieu of a tracer fluid, a radioopaque or isotopic solution can be injected. An x-ray or radiation detecting machine is used to confirm the proper penetration of the tool.

While the preferred embodiment of the invention has been described, modifications can be made and other embodiments may be devised without departing from the spirit of the invention and the scope of the appended claims.

What is claimed is:

1. A device for the treatment of a patient's canaliculus and nasolacrimal duct stenosis which comprises:
a tube shaped and dimensioned to penetrate said canaliculus and duct;
said tube being made of a substantially hard material, and having a proximal end, a blunted distal end, an axial lumen, a total length between approximately 4 and 50 centimeters and an outer diameter between 0.125 and 4.00 millimeters;
a connector at said proximal end;
said lumen having at least one orifice at said distal end.

2. The device of claim 1, wherein said orifice comprises at least one radial outlet about 0.5 to 30 millimeters from said distal end, and said outlet has a diameter between about 0.025 and 2.5 millimeters.

3. The device of claim 1, wherein said material substantially is taken from a group consisting essentially of stainless steel, bronze, silver, aluminum, titanium, brass, and alloy thereof, Kevlar, Nitinol, polymide, Dacron, nylon, EPTFE and PVC; and said tube further comprises a slanted radial flange proximate to said connector.

4. The device of claim 1 which further comprises a flexible catheter having a distal end shaped and dimensioned to interlock with said connector, and a proximal end shaped and dimensioned to interlock with a syringe.

5. The device of claim 4, wherein said connector is shaped and dimensioned to interlock with a syringe.

6. The device of claim 1 which further comprises a stiffening rod diametrically sized to engage said lumen, and having a length at least equal said total length.

7. The device of claim 6, wherein said rod has an enlarged manipulable end section.

8. The device of claim 1 which further comprises:
a flexible sleeve having a proximal end, a distal end, an axial interior channel closed at said distal end and being dimensioned to allow said channel to be engaged by said tube, and a length shorter than said total length of said tube.

9. The device of claim 8, wherein said flexible sleeve has a radial hole proximate said distal end.

10. The device of claim 8, wherein said tube comprises a radial flange proximate to said connector, and wherein said sleeve comprises a first radial flange around said proximal end; said first radial flange being oriented at the same axial angle as the radial flange of said tube.

11. The device of claim 10 wherein said sleeve comprises a second radial flange distally proximate to said first radial flange.

12. The device of claim 1, which further comprises:
a flexible sleeve having a proximal end, a distal end, an axial interior channel closed at said distal end;
a sheath shaped and dimensioned to friction fit within said interior channel and near said distal end of said flexible sleeve; and,
said sheath being further shaped and dimensioned to define an inner hollow well dimensioned to be engaged by said tube.

13. The device of claim 12, wherein said sheath has an axial length shorter than an axial length of said sleeve.

14. The device of claim 9, wherein said sleeve further comprises a first sealing implement across said channel, proximate said proximal end.

15. The device of claim 9, wherein said sleeve further comprises an inflatable segment between said radial hole and said distal end.

16. The device of claim 15, wherein said sleeve further comprises a second sealing implement across said channel at a short proximal distance from said segment.

17. The device of claim 15, wherein said inflatable segment comprises said sleeve having a reduced wall thickness along said segment.

18. A method for probing the integrity of a patient's canaliculus and nasolacrimal duct which comprises the steps of:
inserting the device of claim 1 through the patient's punctum and canaliculus down the lacrimal sac;
tilting the device about 90 degrees into alignment with the nasolacrimal duct;
pushing the device through the nasolacrimal duct down to the nasal cavity;
injecting a tracing fluid through said connector; and
recovering part of said fluid from the nasal cavity;
whereby recovery of a trace of said fluid confirms that the device has penetrated all obstructions and entered the nasal cavity.

19. A method for intubating a patient's nasolacrimal duct which comprises the steps of:
inserting the metallic tube and the sleeve of claim 8 through a patient's punctum, canaliculus into the lacrimal sac;
tilting the sleeve and tube about 90 degrees into alignment with the patient's nasolacrimal duct;
pushing the tube and sleeve through the nasolacrimal duct down to the nasal cavity;
injecting a tracing fluid into the tube;
verifying that the tube and sleeve have reached the nasal cavity by recovering traces of said fluid in said cavity; and
withdrawing said tube from said sleeve.

20. A method for intubating a patient's nasolacrimal duct which comprises the steps of:
inserting the tube and the sleeve of claim 16 through a patient's punctum, canaliculus into the lacrimal sac;
tilting the sleeve and tube about 90 degrees into alignment with the nasolacrimal duct;
pushing the tube and sleeve through the nasolacrimal duct down to the nasal cavity;
injecting a volume of fluid through said connector sufficient to inflate said inflatable segment;
partially withdrawing said tube from said sleeve by a distance sufficient to bring said outlet between said proximal end of the sleeve and said sealing implement at a short proximal distance from the inflatable segment;
injecting a tracing fluid into the tube;
verifying that the tube and sleeve have reached the nasal cavity by recovering traces of said fluid in said cavity; and
withdrawing said tube from said sleeve.

21. The method of claim 18, which further comprises inserting a stiffening rod diametrically sized to engage said lumen and having a length greater than said total length into said tube, prior to insertion of said tube into said sleeve.

22. The method of claim 20, which further comprises inserting a stiffening rod diametrically sized to engage said lumen and having a length substantially greater than said total length into said tube prior to insertion of said tube into said sleeve.

23. The method of claim 18, which further comprises connecting said tube to a suction device during said step of pushing.

24. The method of claim 19, which further comprises connecting said tube to a suction device during said step of pushing.

25. The method of claim 18, wherein said step of recovering comprises connecting a suction device to said tube.

26. The method of claim 19, wherein said step of recovering comprises connecting a suction device to the proximal end of said tube.

27. The method of claim 19, wherein said step of pushing further comprises pushing said second flange inside said punctum and resting said first flange against the external rim of said punctum.

28. A multi-functional surgical tool for the treatment of nasolacrimal obstruction which comprises:
a first elongated tubular conduit having proximal and distal ends;
a second elongated tubular conduit having proximal and distal extremities, said second conduit being parallelly coupled to said first conduit; and
an inflatable balloon at a distal end of said first conduit;
said surgical tool being sized to be introduced into a patient's nasolacrimal duct through one of said patient's canaliculi.

29. The tool of claim 28, wherein said second conduit comprises at least one port near said distal extremity.

30. The tool of claim 28, wherein said coupled conduits have a maximum cross-sectional dimension of between about 1.0 millimeter and 2.5 millimeters.

31. The tool of claim 30, wherein said first and second conduits are coaxial.

32. The tool of claim 31, wherein said second conduit comprises:
a median portion running inside said first conduit and balloon;
a proximal section exiting through an opening in said first conduit; and
a distal section exiting through an aperture beyond said balloon.

33. The tool of claim 32, wherein said opening is sealed around said proximal section and said aperture is sealed around said distal section.

34. The tool of claim 33, which further comprises a first connector at said proximal end and a second connector at said proximal extremity.

35. The tool of claim 34, which further comprises an irrigation device connected to said second connector.

36. The tool of claim 34, which further comprises a suction device connected to said second connector.

37. The tool of claim 34, which further comprises an inflating device connected to said first connector.

38. The tool of claim 34, wherein said second conduit has an angled distal part.

39. A surgical tool which combines a balloon catheter and an irrigation conduit coupled together in an elongated instrument sized to be manually introduced into a patient's nasolacrimal network, wherein an inflatable balloon and a discharge port of the irrigation conduit are located near the distal end of the instrument.

40. A method for treating an obstruction in a patient nasolacrimal system, which comprises:
inserting into said system a tool combining a balloon catheter and an irrigation catheter, joined parallelly to each other;
inflating said balloon catheter; and
injecting a tracer fluid through said irrigation catheter.

41. The method of claim 40, which further comprises detecting an amount of said tracer fluid beyond said obstruction.

42. The method of claim 40, which further comprises successively inflating and deflating said balloon catheter, and performing a plurality of said injecting steps.

43. The method of claim 40, which further comprises suctioning blood out of said system through said irrigation catheter.

44. The method of claim 40, which further comprises injecting medication in said system through said irrigation catheter.

45. The method of claim 40, which further comprise piercing a passageway through said patient's inferomedial wall with a probe and running said tool through said passageway.
